(19) United States
(12) Patent Application Publication
(10) Pub. No.: US 2011/0097709 A1
(43) Pub. Date: Apr. 28, 2011

(54) Method for modifying a nucleic acid

(76) Inventors: Geoffrey L. Kidd, Germantown, MD (US); Colin Higbie, Reva, VA (US)

(21) Appl. No.: 09/805,839

(22) Filed: Mar. 13, 2001

Related U.S. Application Data
(60) Provisional application No. 60/188,805, filed on Mar. 13, 2000.

(51) Int. Cl. C12Q 1/68; C12N 1/21

(52) U.S. Cl. 435/6; 435/252.33

(57) ABSTRACT

Disclosed are methods for increasing the rate at which a nucleic acid can be translated by modifying the polynucleotide such that the rate at which the polynucleotide is translated is altered. Also disclosed are methods for modulating the translation of an mRNA by adding a nucleic acid that increases translation of the mRNA

### RELATED APPLICATIONS

This application claims priority to U.S. Ser. No. 60/188,805, filed Mar. 13, 2000. The contents of this application are incorporated by reference in their entirety.

### BACKGROUND OF THE INVENTION

The invention relates to nucleic acids and polypeptides and more particularly to methods for modifying nucleic acids to modulate expression of genetic information encoded by the nucleic acids.

Several steps are involved in the expression of genetic information as a polypeptide product. A gene encoding a polypeptide is first transcribed into mRNA, which is then translated by ribosomes that move processively along the mRNA. The ribosomes read three-letter nucleotide triplets known as codons in the RNA and assemble appropriate amino acids based on the sequence of a given codon. As the amino acids are assembled, the nascent peptide begins to fold, acquiring secondary and tertiary structure. The structure that forms at a given moment depends upon which amino acids are present in the nascent peptide and available for interaction at that moment.

### SUMMARY OF THE INVENTION

The invention is based in part on the discovery that modulating the structure of mRNA can affect the rate at which polypeptide encoding genetic information is translated into a protein.

In one aspect, the invention provides a method for modifying a polypeptide-encoding nucleotide sequence. A first polypeptide-encoding nucleotide sequence, which includes a plurality of codons encoding a polypeptide sequence, is provided, and a first secondary structure in the polypeptide-encoding nucleotide sequence is determined. At least one nucleotide is then altered in the first polypeptide-encoding nucleotide sequence, thereby producing a second nucleotide sequence, and a secondary structure is determined for the second nucleotide sequence. The first and second secondary structure and the second secondary structure, thereby modifying a polypeptide-encoding nucleotide sequence.

In some embodiments, the second nucleotide sequence contains at least one region that differs from the corresponding region of the first polypeptide-encoding nucleotide sequence.

In some embodiments, the first secondary structure and second secondary structure differ in stability, e.g., the second secondary structure is more stable or less stable then the first secondary structure. For example, alteration of the nucleotide may produce a secondary structure that contains altered base-pairing of a nucleotide sequence in at least one region of the second nucleotide sequence relative to the corresponding region in the first polypeptide-encoding sequence. The first and second nucleotide sequences can differ in secondary structures over either a small region or a large region. For example, the region can be, e.g., 5-1000 nucleotides or 10-500, 10-250, 20-125, or 25-75 nucleotides.

In preferred embodiments, the altered nucleotide is in a codon of the first polypeptide-encoding polynucleotide. For example, the altered nucleotide may alter (by either increasing or decreasing) the number of cytosine and guanine nucleotides in the codon in the second nucleotide sequence as compared to the codon in the first polypeptide-encoding nucleotide sequence. In some embodiments, multiple codons are altered in the first polypeptide-encoding nucleotide sequence. For example, the alteration can change at least 2, 5, 10, 15, 25, 50, 100, or more codons in the first polypeptide-encoding sequence.

In preferred embodiments, the second nucleotide sequence encodes a polypeptide having the same polypeptide sequence as the polypeptide sequence encoded by the first polypeptide-encoding nucleotide sequence. In other embodiments, the second nucleotide sequence encodes polypeptide sequences having conservative amino acid substitutions in its amino acid sequences relative to the amino acid sequence encoded by the first nucleotide sequence.

The polypeptide-encoding nucleotide sequence can be either DNA or RNA.

In another aspect, the invention features a method for modifying a polypeptide-encoding nucleotide sequence by providing a first polypeptide-encoding nucleotide sequence from a first organism, wherein the polypeptide-encoding nucleotide sequence includes a plurality of codons encoding a polypeptide sequence and identifying the frequency at which a first codon of the first polypeptide-encoding nucleotide sequence occurs in polypeptide-encoded genes of the first organism. At least one nucleotide in the first codon is altered, thereby producing a second nucleotide sequence including a first replacement codon. The first replacement codon occurs at a different frequency in polypeptide-encoded genes of the first organism than the first codon. In some embodiments, the first replacement codon occurs at a lower frequency in polypeptide-encoding genes of the first organism than the first codon. In some embodiments, the first replacement codon occurs at a higher frequency in polypeptide-encoding genes of the first organism than the first codon.

Preferably, the first replacement codon encodes an amino acid identical to the amino acid encoded by the first codon.

In some embodiments, the method further includes identifying the frequency at which a second codon of the first polypeptide-encoding nucleotide sequence occurs in some or all of the polypeptide-encoded genes of the first organism, and replacing at least one nucleotide in the second codon to produce a second nucleotide sequence including a second replacement codon. The second replacement codon occurs at a different frequency in polypeptide-encoded genes of the first organism than the first codon.

In some embodiments, the second codon is adjacent to the first codon in the first polypeptide-encoding polynucleotide sequence.

Preferably, the second nucleotide sequence encodes an RNA molecule translated at a different rate than an RNA molecule encoded by the first polypeptide-encoding nucleotide sequence. For example, the second nucleotide sequence can encode an RNA molecule that is translated more rapidly than the first polypeptide-encoding nucleotide sequence. Alternatively, the second nucleotide sequence encodes an RNA molecule that is translated more slowly than the first polypeptide-encoding nucleotide sequence.

In preferred embodiments, the method includes identifying the frequency at which the first codon occurs in some or all of the polypeptide-encoded genes of a second organism and replacing at least one nucleotide in the first codon to produce a first replacement codon. Preferably, the second codon occurs at a similar frequency in the second organism as the first codon occurs in the polypeptide-encoded genes of the first organism.

In another aspect, the invention provides a method for modifying a polypeptide-encoding nucleotide sequence. A first polypeptide-encoding nucleotide sequence, which encodes a plurality of codons encoding a polypeptide sequence is provided, and the cytosine-guanine content, i.e., the number of guanine and cytosine nucleotides, in the codon is determined. At least one nucleotide in the first codon is replaced to produce a second nucleotide sequence including a first replacement codon. The first replacement codon has a guanine-cytosine content different than the first codon. Preferably, the first codon and the first replacement codon encode the same amino acid.

Preferably, the second polynucleotide sequence encodes an RNA molecule translated at a rate different than an RNA molecule encoded by the first polynucleotide sequence.

In preferred embodiments, the method further includes identifying the guanine-cytosine content of a second codon in the polypeptide-encoding nucleotide sequence and replacing at least one nucleotide in the second codon to produce a second nucleotide sequence including a second replacement codon. The second replacement codon has a guanine-cytosine content different than the second codon. Preferably, the second replacement codon and the second codon encode the same amino acids. Any additional number of codons can be monitored. For example, in additional embodiments, three, four, five or more of the codons in the first polypeptide-encoding sequence are altered. The second and subsequent altered codons can be adjacent to the first altered codon, or can be separated by unaltered codons.

Also within the invention is a method for constructing a nucleic acid for increasing expression of a polypeptide-encoding nucleotide sequence. The method includes identifying codon frequencies of a polypeptide-encoding nucleotide sequence and codon frequencies in one or more polypeptide-encoded genes of a first cell and comparing the codon frequencies, thereby identifying at least one rare codon that is present in the transgene and occurs in low frequency in polypeptide-encoded genes of the cell. A construct is then prepared that includes at least one tRNA gene with an anticodon for the rare codon. Preferably, the construct is an episomal vector that replicates autonomously from the endogenous genome of the host cell. The episomal preferably includes additional sequence elements that allow for replication of, and selection for, the episome in the host cell.

If desired, codon frequencies for a second rare codon and additional codons (e.g., three, four, five, six, or ten or more genes) can be identified, and tRNA genes with an anticodon for the second rare codons added to the construct. Alternatively, the tRNA genes with additional codons can be provided as separate constructs in the cell.

In some embodiments, the host cell is a prokaryotic cell, e.g., an E. coli cell.

Also provided by the invention are constructs (such as episomal constructs) and cells containing the constructs made by the herein described methods.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and are not intended to be limiting.

Other features and advantages of the invention will be apparent from the following detailed description and claims.

### BRIEF DESCRIPTION OF THE DRAWINGS

FIG. 1A is a schematic illustration of an endostatin mRNA showing regions of secondary structure.

FIG. 1B is a schematic illustration of a modified endostatin mRNA showing regions of secondary structure. The endostatin mRNA has been modified to have decreased GC-content.

### DETAILED DESCRIPTION OF THE INVENTION

The invention provides methods for modifying nucleic (e.g., polypeptide-encoding nucleotide sequences) so as to optimize their expression, or the expression of another gene or genes, in a host cell of interest.

The methods described are based in part on modulating nucleic acid structure to affect translation of its cognate mRNA. In general, a structure forming from a string of, for example, ten amino acids at a given moment may be quite different from that which would form if five additional amino acids were present on the nascent peptide at that moment. A ribosome translating at faster rate will assemble a longer peptide in a given time interval than a ribosome translating at a slower rate. The longer, faster-growing peptide, having more amino acids than the shorter, slower-growing peptide at any given moment, may therefore fold in a different way and assume a different structure because its amino acid content at that moment is different from that of the slower growing peptide.

In one embodiment, codons of genes are altered such that a secondary structure is removed or reduced without changing the amino acid sequence of the encoded polypeptide. The altered codons are preferably selected so that that a secondary structure or structures (e.g., as expressed in a particular base-pairing pattern) are decreased in strength or removed completely.

Polynucleotide-encoding sequences can also be modified by altering nucleotide sequences to introduce relatively rare or abundant codons (relative to codon frequency in the host cell in which the polypeptide-encoding sequence will be expressed) in order to decrease, or increase, the synthesis of the encoded polypeptide during translation. Modulating translation in this manner allows for modulation of the rate at which the nascent polypeptide folds. While not wishing to be bound by theory, it is believed that the nascent polypeptide structure that forms at a given moment in translation depends upon which amino acids are present in the nascent peptide and available for interaction at that moment. A structure forming from a peptide of 10 amino acids may be quite different from that which would form if the peptide includes five additional amino acids. A ribosome translating at a faster rate will assemble a longer peptide in a given time interval than a ribosome translating at a slow rate. The longer, faster-growing peptide may therefore fold in a different way and assume a different structure because its amino acid content differs from that of the slower growing peptide.

In some cases, the rate at which a ribosome translates mRNA is believed affected by codon content. A preferred codon for a given host cell can be considered to be one found at a high frequency in a particular host cell, or in a class of genes, relative to the other codons for the same amino acid. Thus, a preferred codon for a given host cell may be a codon that occurs with reduced frequency in the cell's poorly expressed genes. Typically, the cognate tRNA species of a codon occurring at high frequency in a cell is believed to be present at high levels relative to the other tRNA species for that amino acid. Translation may therefore proceed through such a codon relatively easily. A codon whose cognate tRNA is present at relatively low levels may not be translated as readily, since it must wait longer for the appropriate tRNA to arrive at the reaction site. Such relatively infrequent codons tend to occur infrequently in a cell's genome, presumably because in most cases quick, efficient translation is more conductive to the cell's survival than slow translation. Thus, the rarity of a codon can determine the speed with which it is translated and, therefore, the amino acids available in the nascent peptide at any given moment for secondary structure formation. Therefore, if a longer or shorter time is desired for protein folding, the codon content of the corresponding gene or gene segment can be adjusted such that the codon frequencies are lower or higher, respectively.

In one embodiment, a polypeptide-encoding gene is transferred from its native cell type to a heterologous host cell, which has a different codon frequency in one or more of the amino acids encoded by the gene. In the gene's new host, the various segments of the gene's messenger RNA may be translated at rates different from the rates in its native environment. These altered translation rates may result in altered folding of the gene's polypeptide product, which may cause the polypeptide to be defective. To obviate this problem, the gene can be redesigned so that it has codons whose individual frequencies in the new host match those of the original codons in the original host. Preferably, the encoded amino acid sequence of the modified nucleotide sequence is unchanged.

Also provided is a method of regulating protein expression in a gene by replacing codons with one GC-content with codons having a differing GC-content. The guanine+cytosine (GC) content of a gene can influence the expression of a gene. One mechanism by which this is believed to occur is by affecting the degree of secondary structure with the gene's cognate mRNA molecules. Because GC base pairs are stronger than adenine-thymine (AT) base pairs, an RNA strand with a high GC content tends to form stronger secondary structures than one with a high AT content. Strong secondary structures within a mRNA molecule may inhibit the progress of ribosomes along it length during translation. Thus, the substitution of AT-rich codons for GC-rich codons within a gene, preferably without changing the encoded amino acids, allows for reduction to or elimination of secondary structure formation with the cognate mRNA. This allows for ribosomes to more easily traverse the mRNA, resulting in more efficient protein production.

Also featured by the invention is a method for facilitating expression of a gene in a host cell. The method includes constructing a vehicle or construct that contains one or more tRNA genes encoding tRNAs cognate to rare, suboptimal, and/or other codons whose occurrence or arrangement within an mRNA molecule causes a slowing of translation of the mRNA molecule into polypeptide. The construct can be used to increase the translation rate of one or more mRNA species in the cell.

The method is suitable for applications in which it is desirable to transfer a gene from a cell in which it is normally expressed, to a second, heterologous cell. Frequently, such a transplanted gene (“transgene”) does not produce desired levels of a polypeptide gene product in the heterologous cell. The decreased expression can arise if a codon is abundant in one cell type but is not abundant in the second cell type. An abundant codon is a codon found at high frequency in a particular cell type, or in a class of genes, relative to other codons for the same amino acid. The intracellular concentration of a given tRNA species can influence whether its cognate codon can be easily, and therefore quickly, translated. Since the relative concentrations of the various tRNA species can vary greatly between cell types, codon proference can vary accordingly. A codon preferred by one cell type can often be a non-preferred codon in a second cell type. In the second cell type, the mRNA may as a result be inefficiently translated.

In addition to transgenes, genes native to a particular cell may also contain rare codons that limit the translation rate of the gene's mRNA.

Translation of an mRNA may also be slowed by the occurrence of several identical preferred codons in tandem within the mRNA. In this arrangement, translation of repeat codons results in local exhaustion of the cognate tRNA.

The present invention provides for an increase in the translation rate of an mRNA species by increasing the intracellular concentration of the tRNA species that are in short supply (“rare tRNAs”) or locally exhausted. This is performed by transferring additional genes for those tRNA species into the cell. The genes are preferably transferred on an autonomously replicating construct, such as plasmid. The plasmid is introduced into the host cell containing the transgene, and the tRNA genes transcribed from the plasmid increase levels of tRNAs (such as rare or otherwise underrepresented tRNAS), thereby allowing rare codons to be translated more quickly. Other vehicles bearing tRNA genes include, e.g., viral, cosmid, and artivial chromosome constructs.

Secondary structures can be calculated based on hypothetical or empirically determined structures. Methods for calculating secondary structures are described or summarized in methods described in, e.g., Zuker, Curr Opin Struct Biol 2000 June; 10(3):303-10; Suhnel, Trends in Genetics 13:206-07, 1997, and RNA Biochemistry and Biotechnology; J. Barciszewski and B. F. C. Clark, Eds., Kluwer Academic Publishers, Dordecht, 1998. Codon frequencies are available for a variety of organisms and are available from sources describe or summarized in, e.g., Nakamura et al., Nucl. Acids. Res. 28:292, 2000.

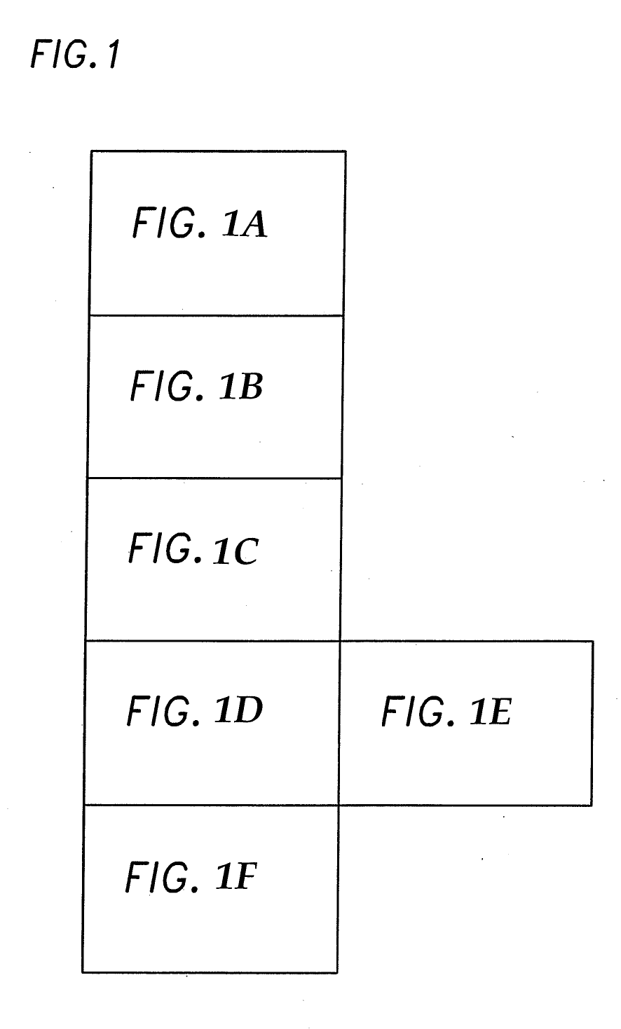

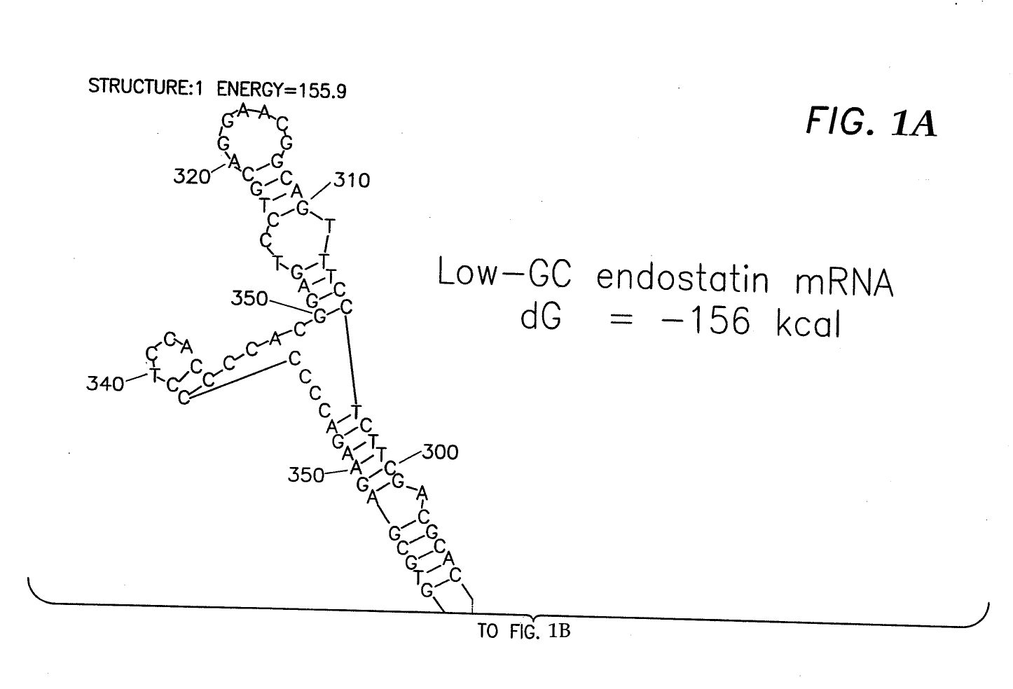

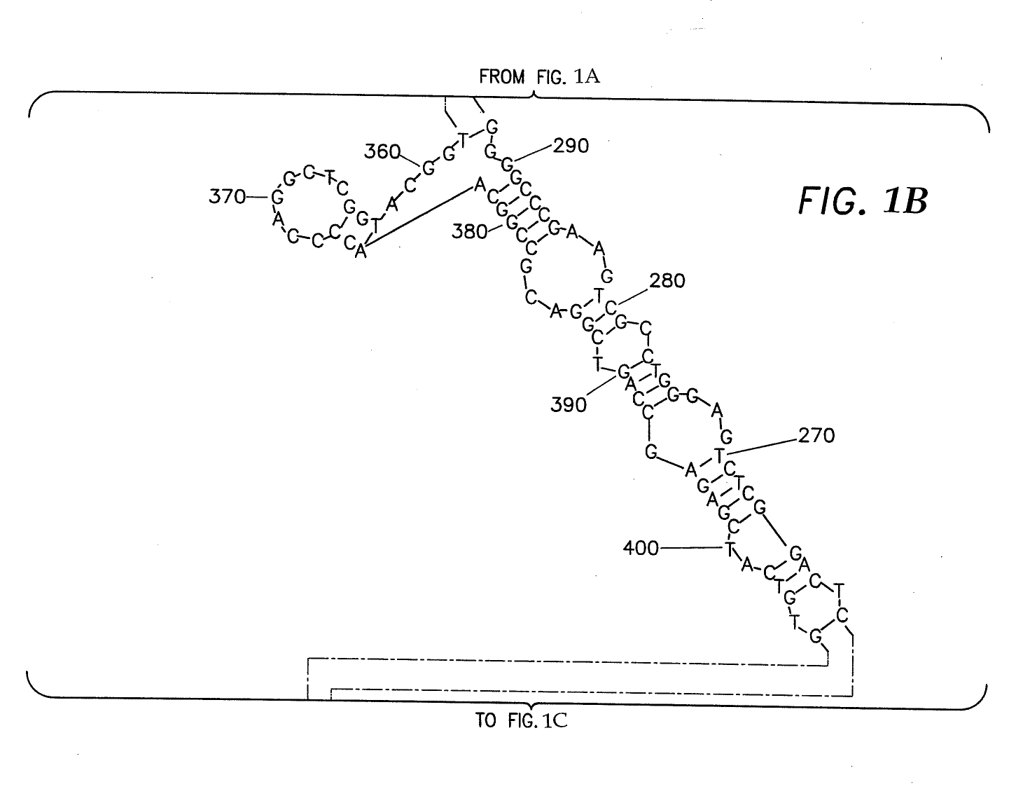

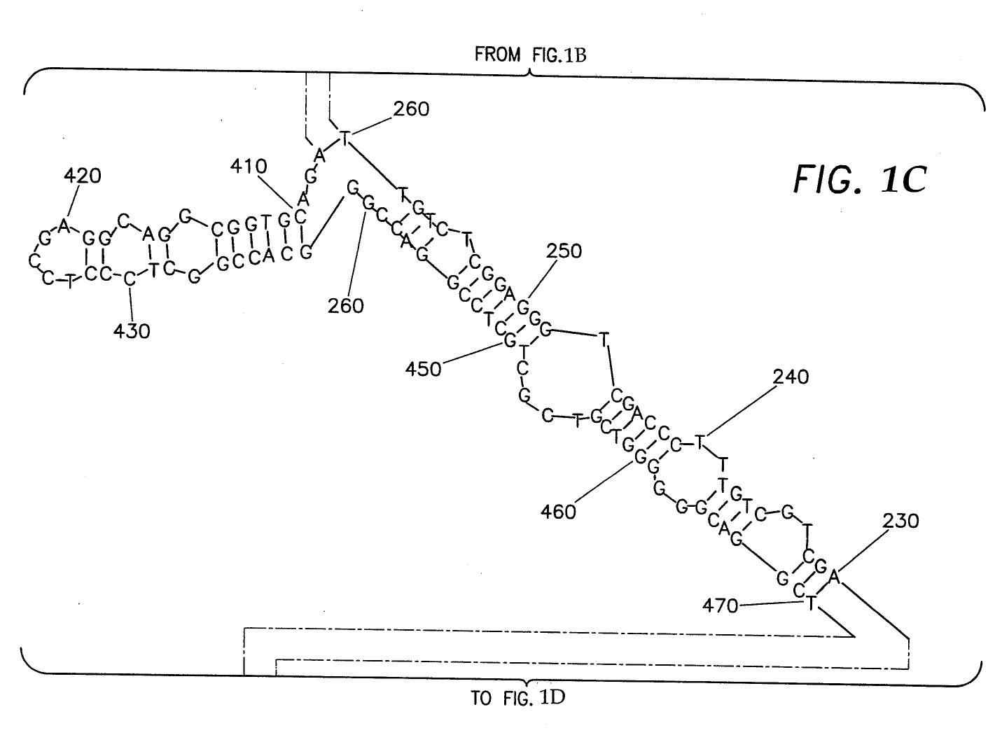

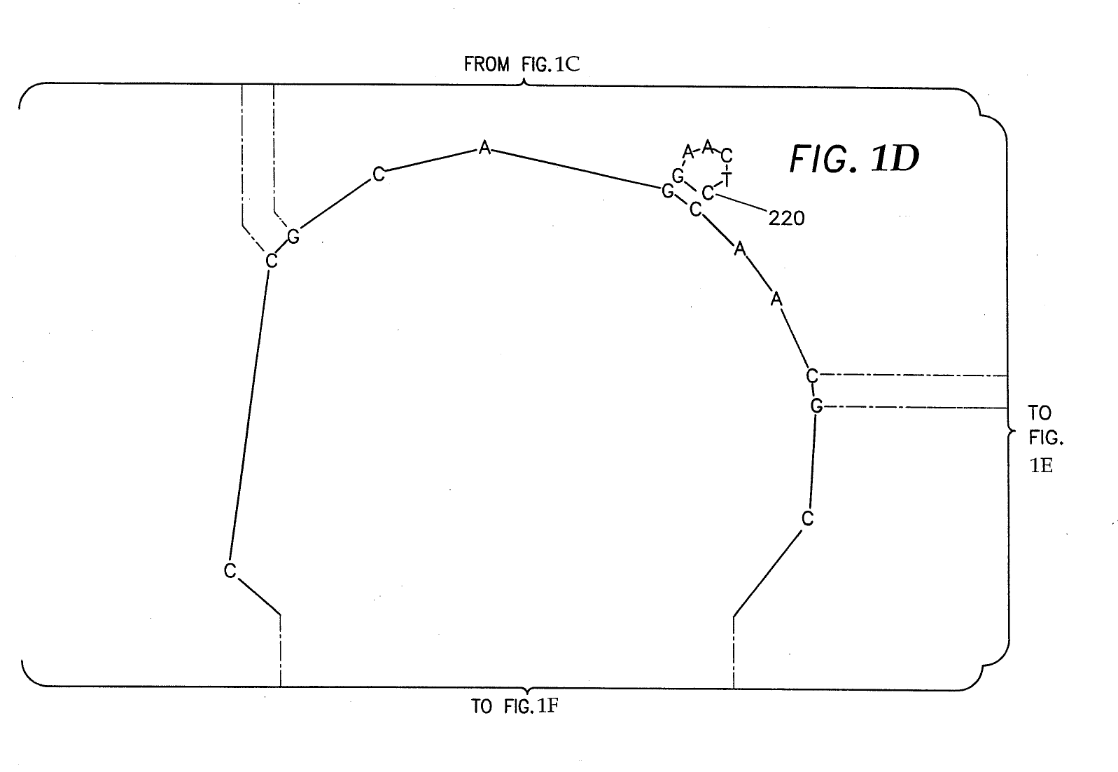

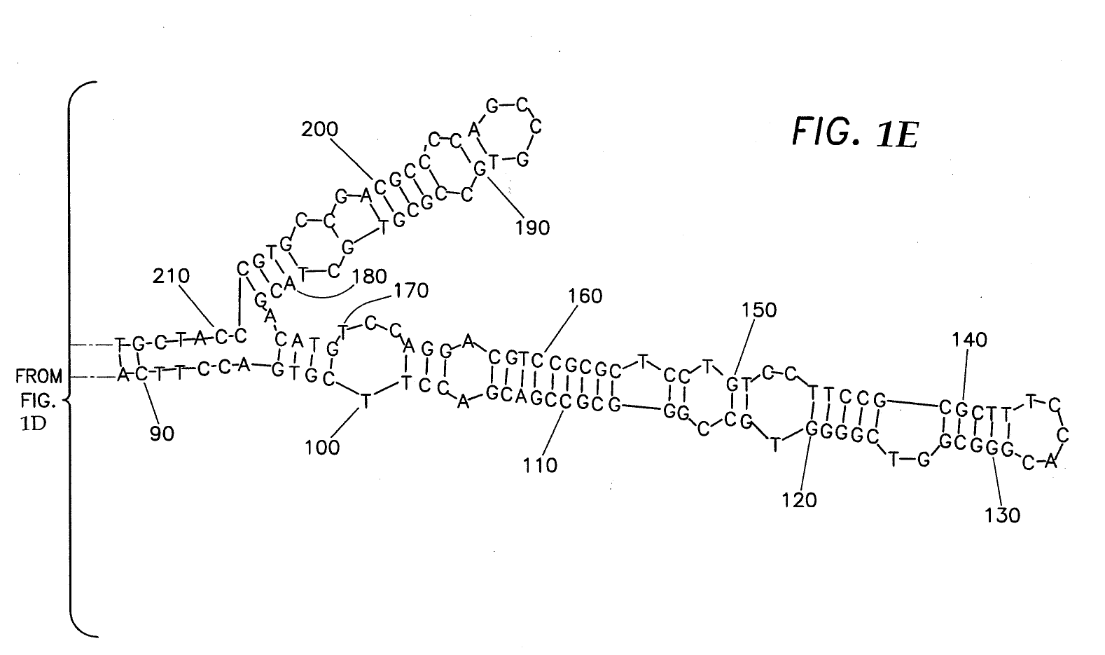

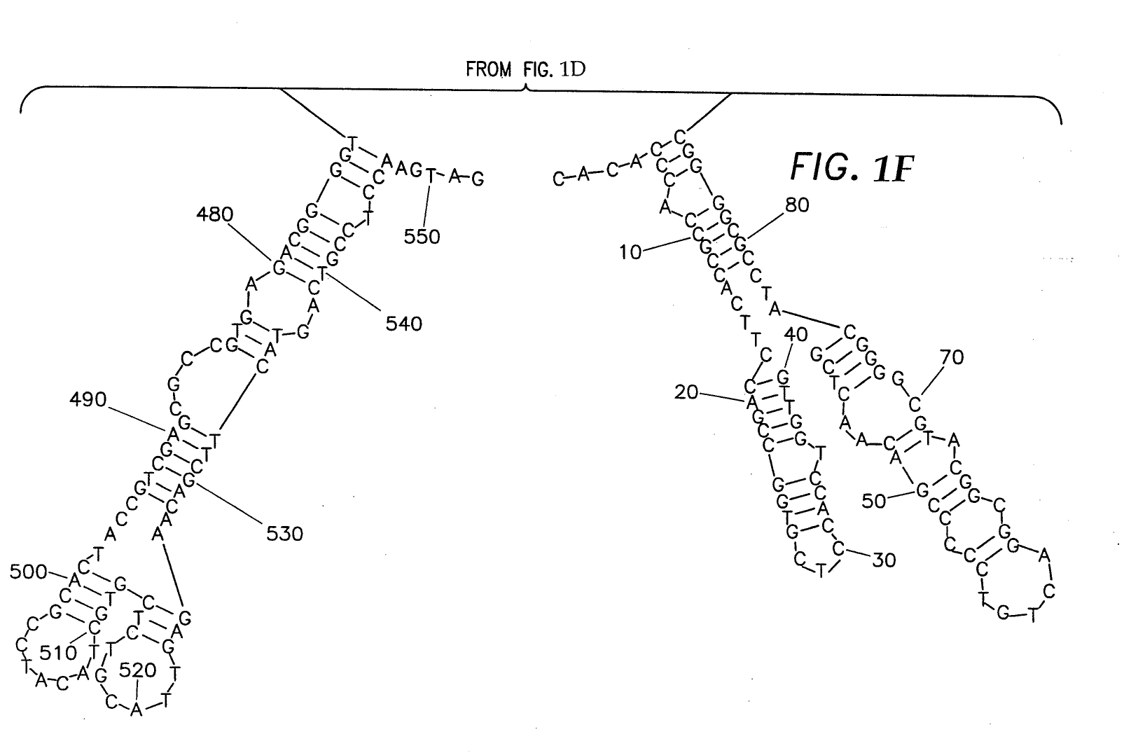

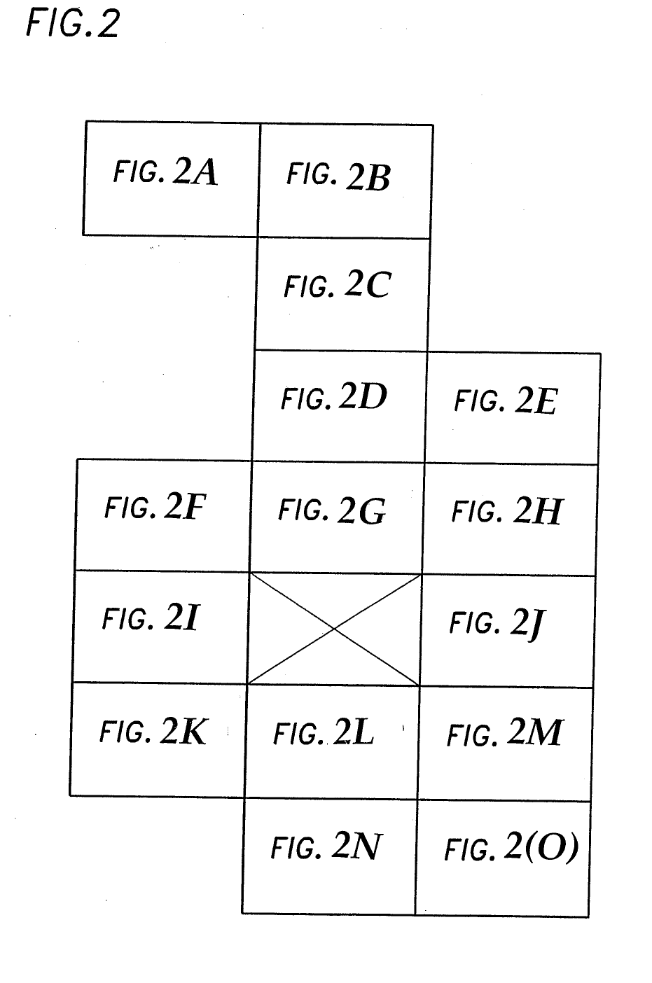

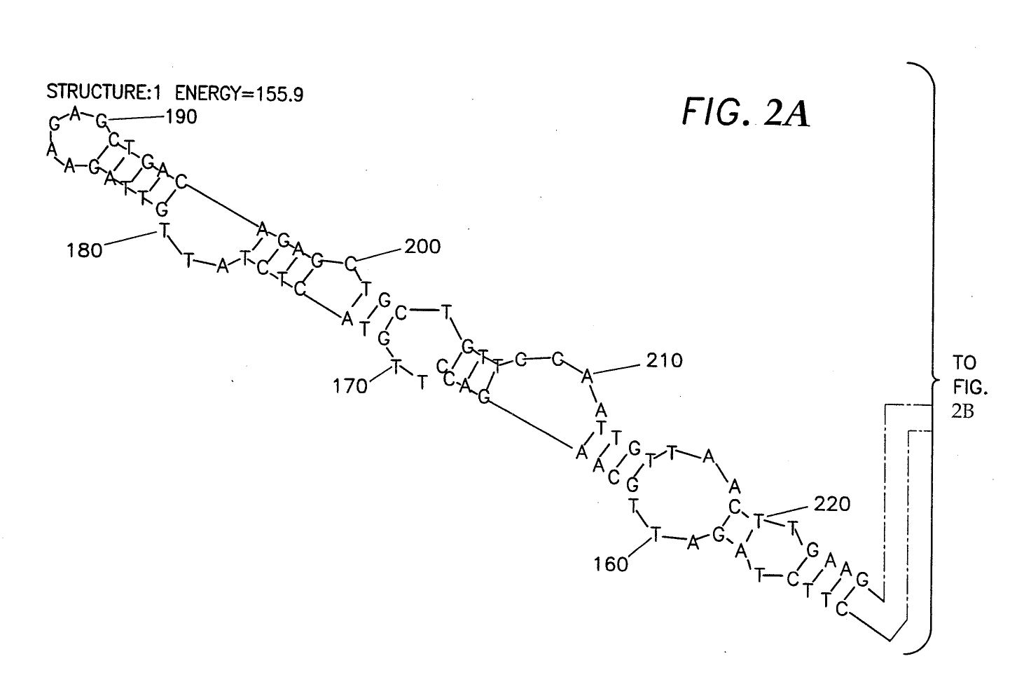

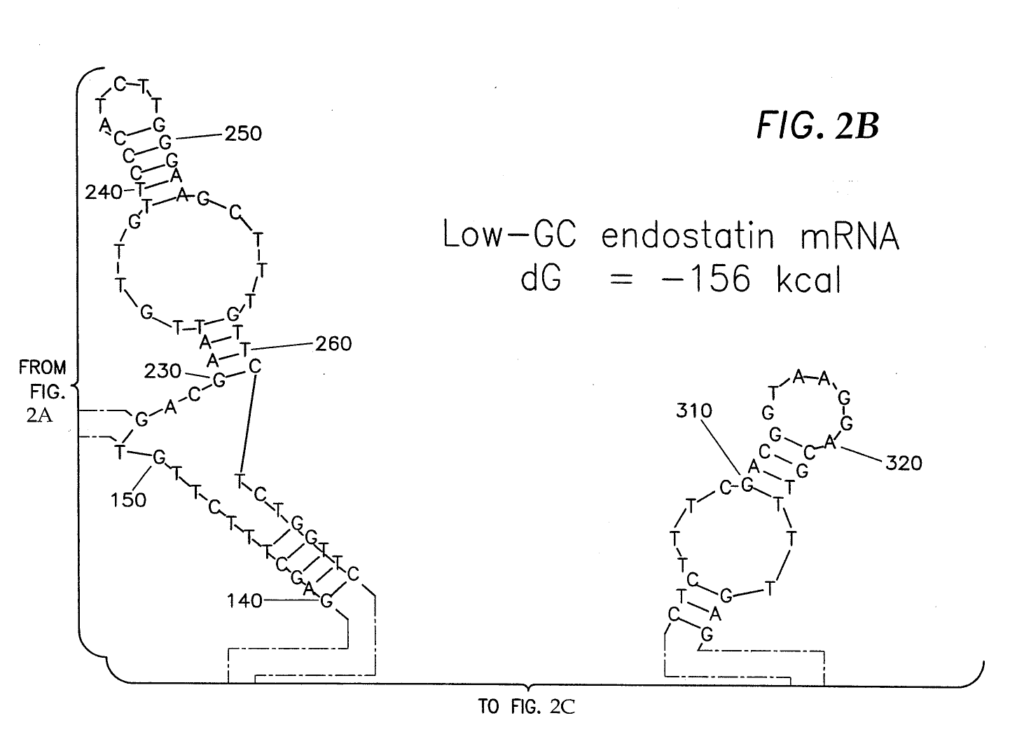

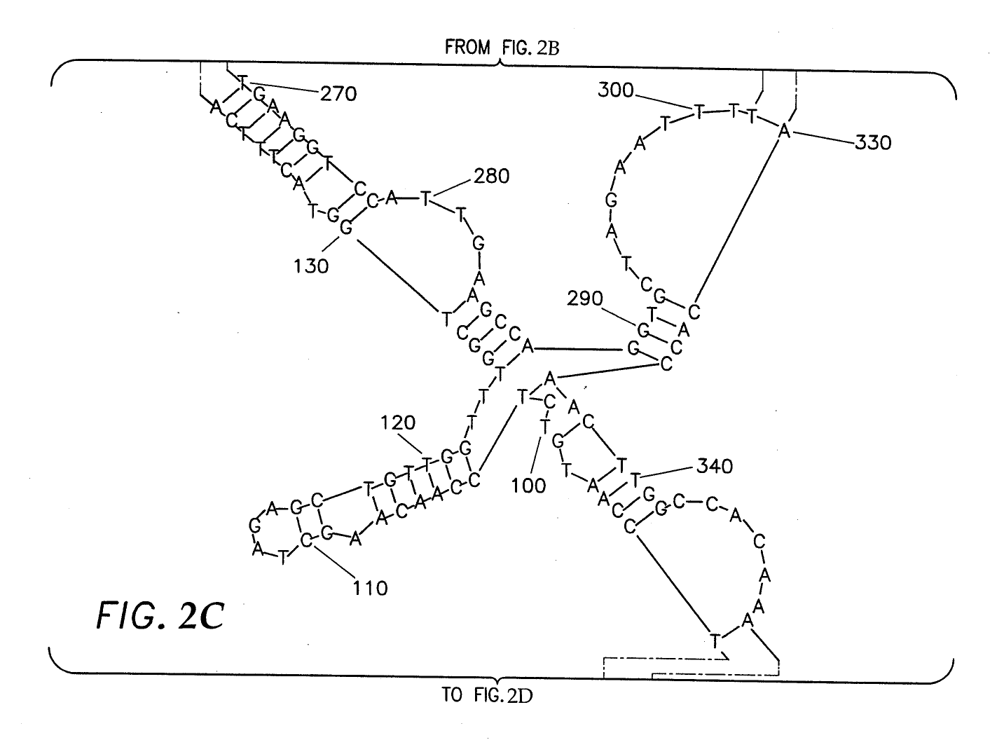

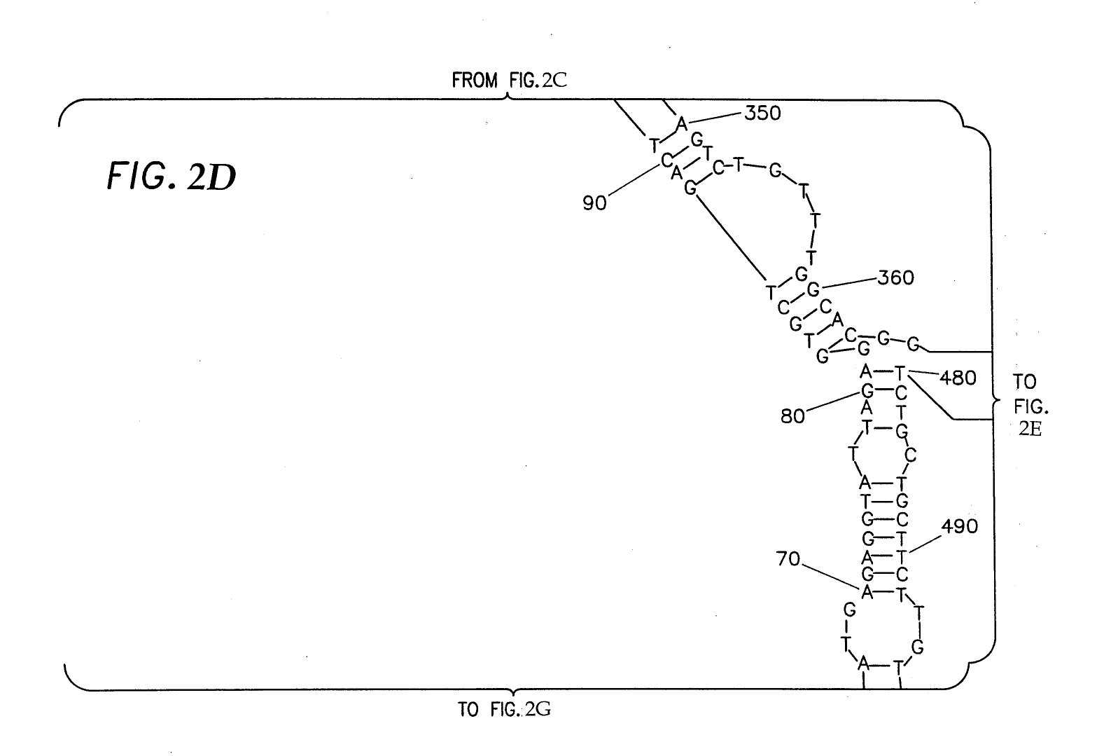

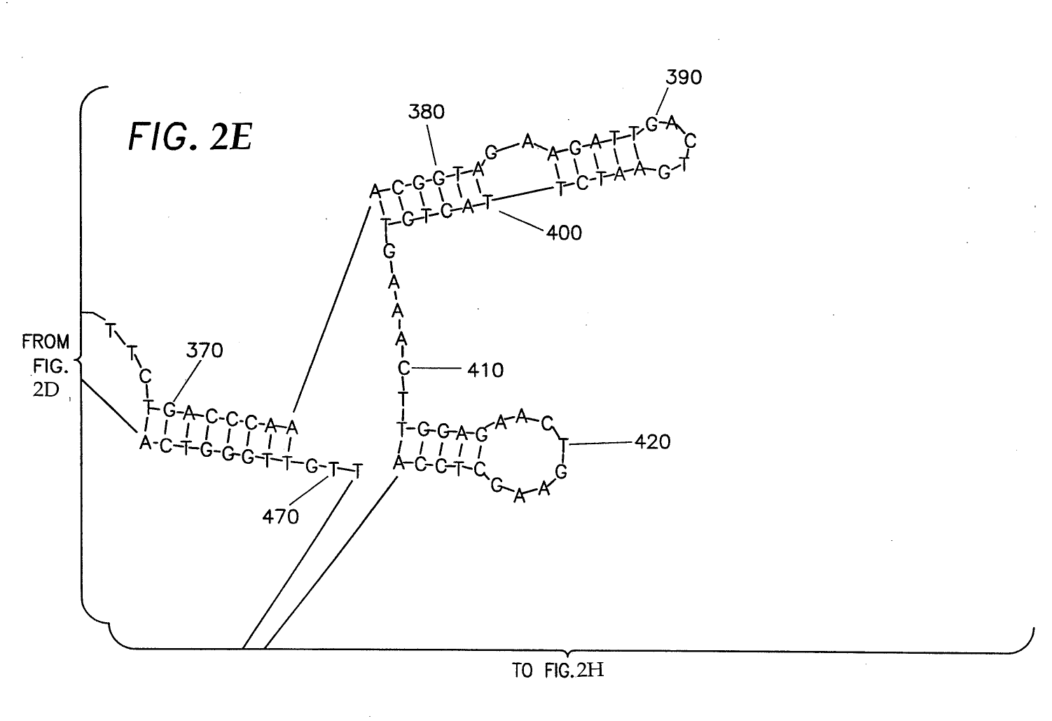

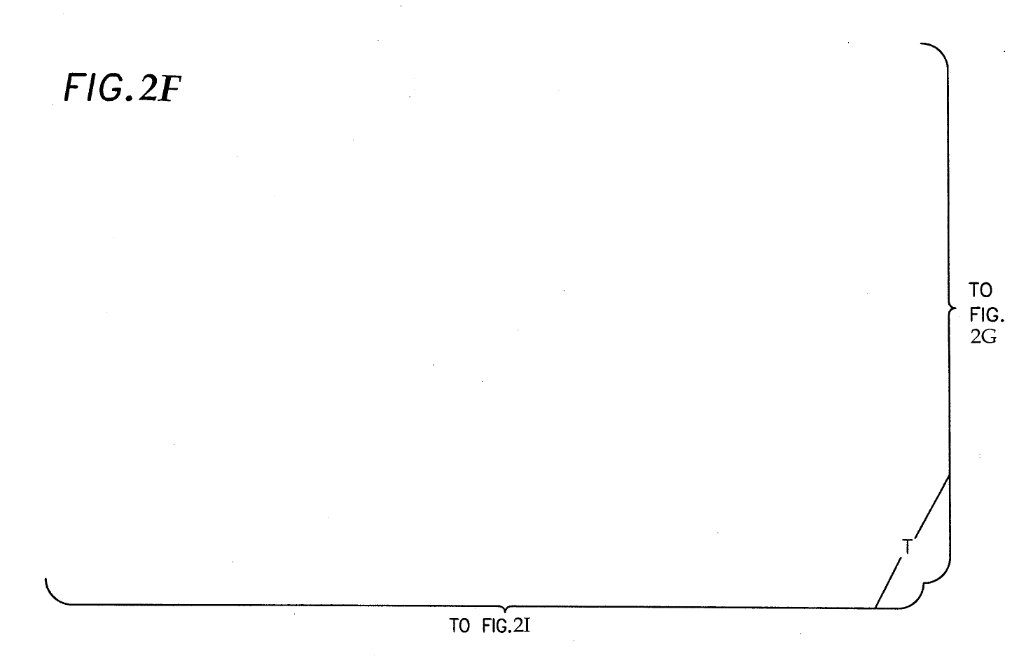

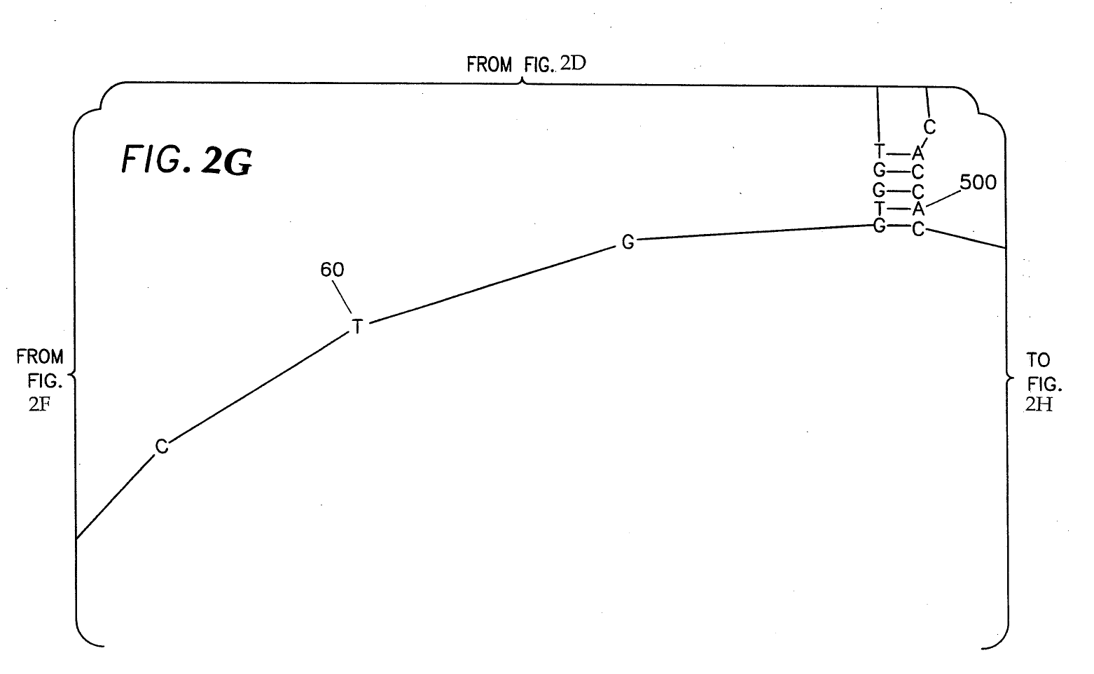

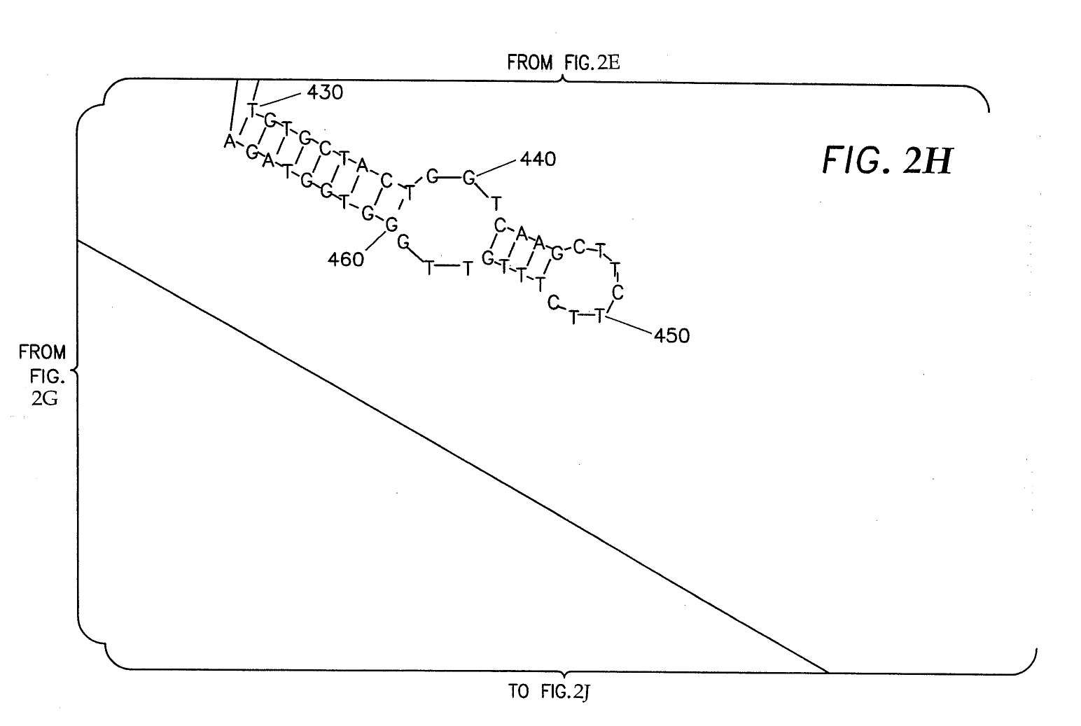

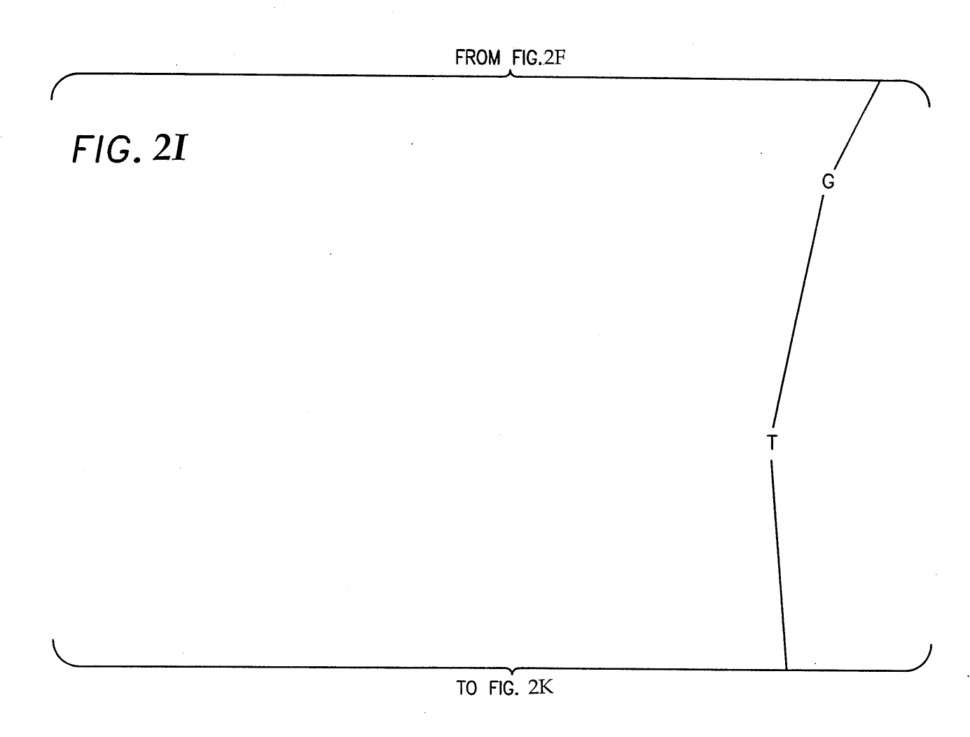

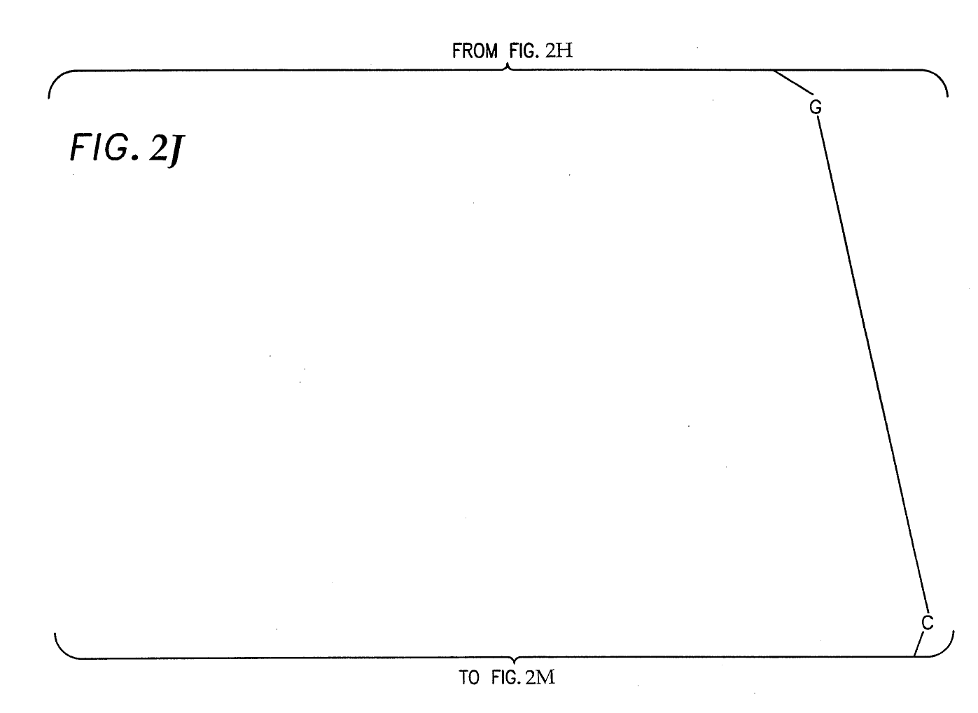

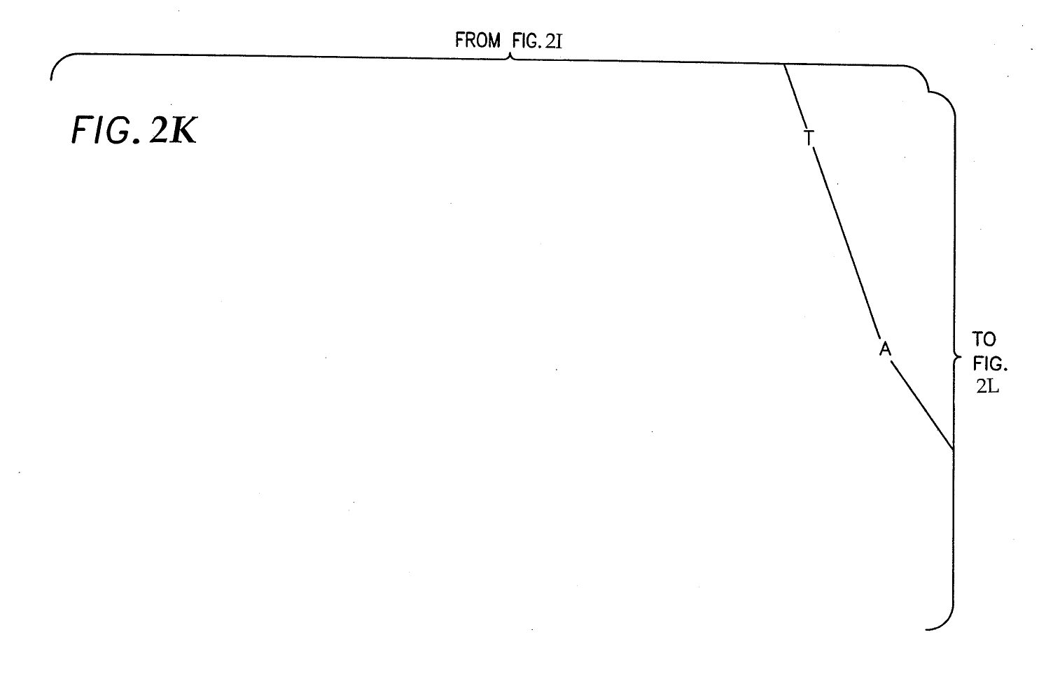

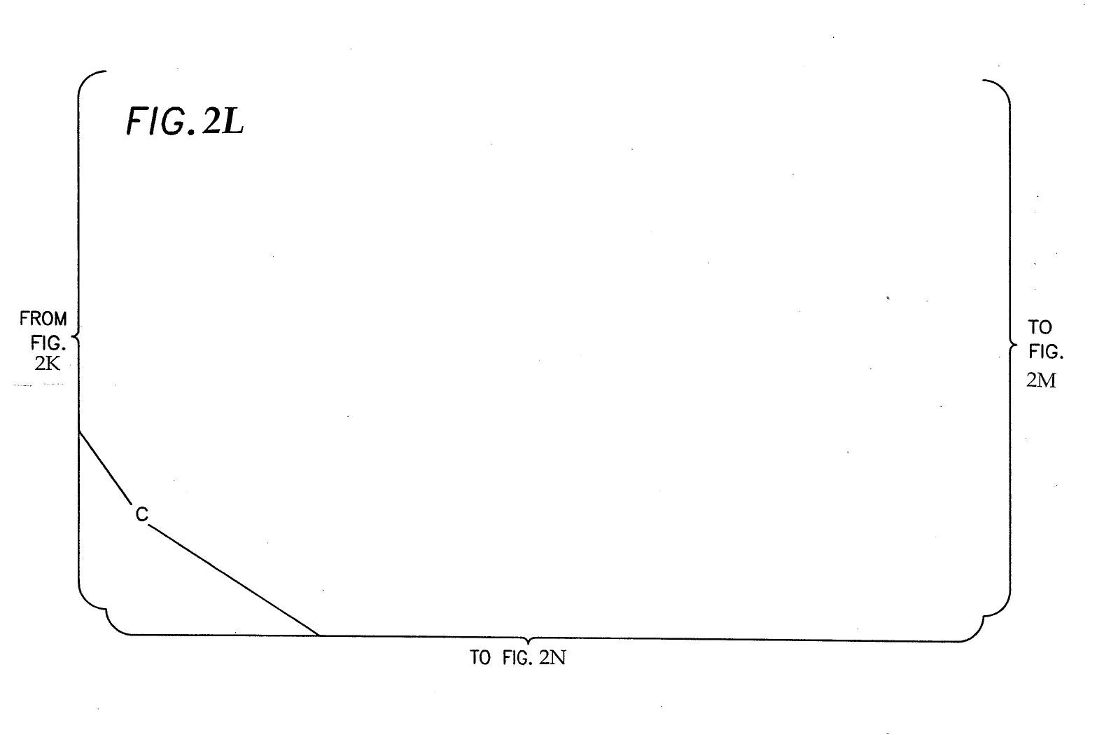

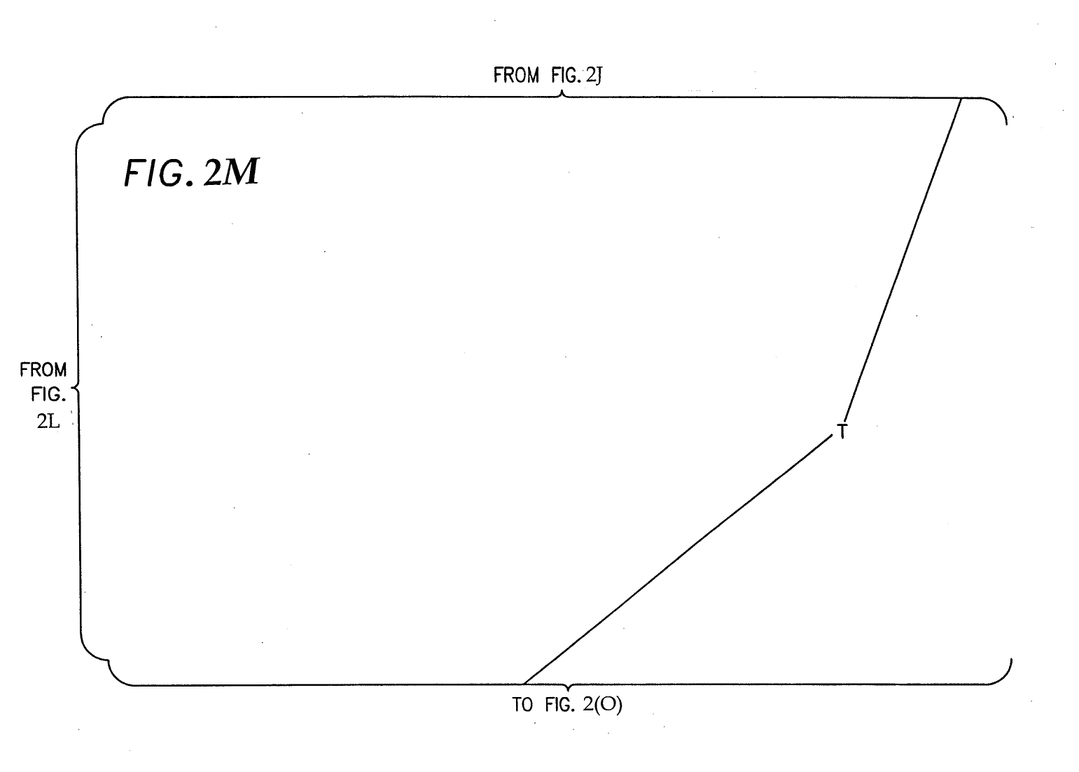

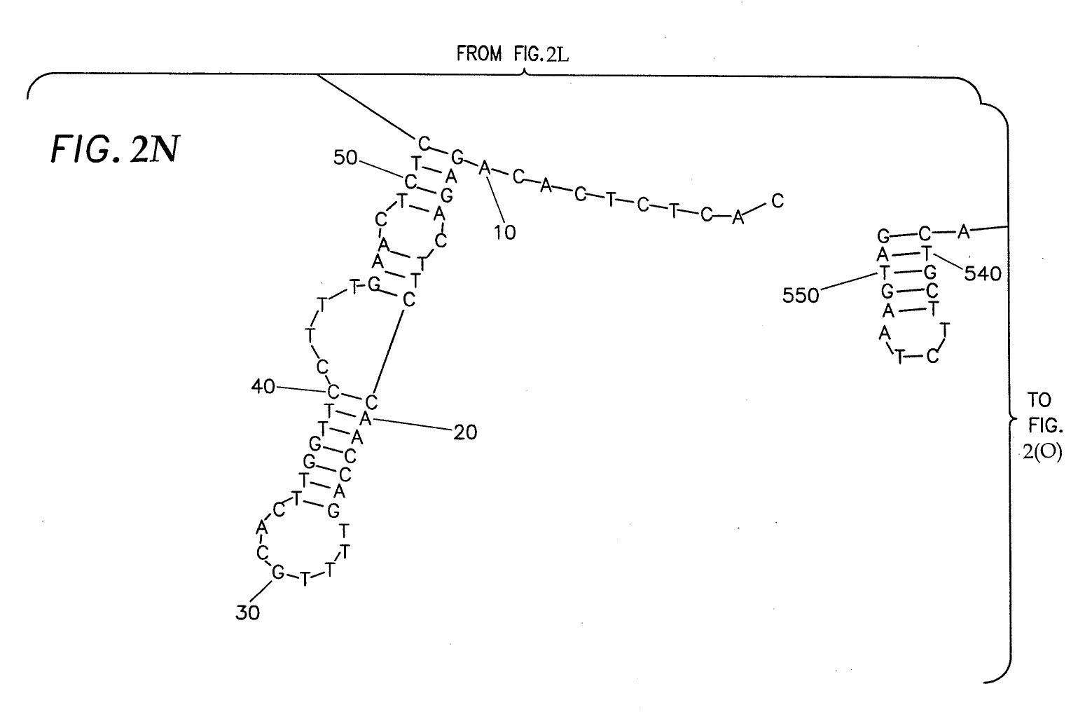

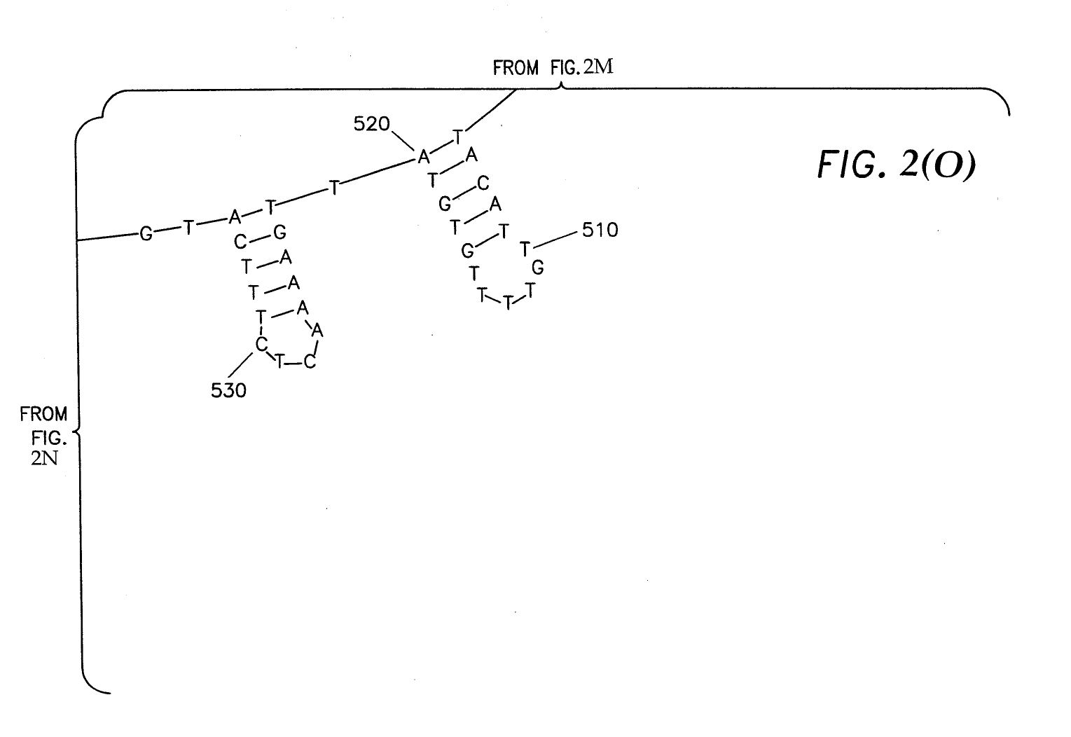

An example of polypeptide-encoding nucleotide sequence redesigned to have an altered secondary structure is shown in FIGS. 1A and 1B. FIG. 1A provides a schematic illustration of an unmodified endostain mRNA sequence showing secondary structures characaterized by regions of base-paired sequences. The free energy (ΔG°) for the predicted structure is −242 kcal. A schematic illustration of endostatin mRNA modified to contain reduced secondary structure while encoding the same polypeptide sequence is shown in FIG. 1B. The ΔG° for the modified structure is −156 kcal.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed:

1. A method for modifying a polypeptide-encoding nucleotide sequence, the method comprising
providing a first polypeptide-encoding nucleotide sequence, wherein said polypeptide-encoding nucleotide sequence includes a plurality of codons encoding a polypeptide sequence;
identifying a first secondary structure for said first polypeptide-encoding nucleotide sequence;
altering at least one nucleotide in said first polypeptide-encoding nucleotide sequence, thereby producing a second nucleotide sequence;
identifying a second secondary structure for said second nucleotide sequence; and
comparing said first secondary structure and said second secondary structure, thereby modifying a polypeptide-encoding nucleotide sequence.

2. The method of claim 1, wherein said second secondary structure is different than said first secondary structure.

3. The method of claim 2, wherein the second secondary structure is more stable than the first secondary structure.

4. The method of claim 2, wherein the second secondary structure is less stable than the first secondary structure.

5. The method of claim 2, wherein base-pairing in at least one region of the second nucleotide sequence is altered relative to the corresponding region in the first polypeptide-encoding sequence.

6. The method of claim 5, wherein said region is 5-105 nucleotides.

7. The method of claim 5, wherein said region is 15-85 nucleotides.

8. The method of claim 5, wherein said region is 25-75 nucleotides.

9. The method of claim 1, wherein said at least one altered nucleotide is in a codon of said first polypeptide-encoding polynucleotide.

10. The method of claim 9, wherein said at least one altered nucleotide alters the number of cytosine and guanine nucleotides in said at least one altered codon.

11. The method of claim 10, wherein said at least one altered nucleotide results in an increased number of cytosine and guanine nucleotides in said codon.

12. The method of claim 10, wherein said at least one altered nucleotide results in an decreased number of cytosine and guanine nucleotides in said codon.

13. The method of claim 10, wherein said alteration changes at least two codons in said first polypeptide-encoding sequence.

14. The method of claim 10, wherein said alteration changes at least five codons in said first polypeptide-encoding sequence.

15. The method of claim 10, wherein said alteration changes at least ten codons in said first polypeptide-encoding sequence.

16. The method of claim 10, wherein said alteration changes at least 50 codons in said first polypeptide-encoding sequence.

17. The method of claim 1, wherein said first polypeptide-encoding nucleotide sequence is DNA.

18. The method of claim 1, wherein said first polypeptide-encoding nucleotide sequence is RNA.

19. The method of claim 1, wherein said second nucleotide sequence encodes a polypeptide having the same polypeptide sequence as the polypeptide sequence encoded by the first polypeptide-encoding nucleotide sequence.

20. A method for modifying a polypeptide-encoding nucleotide sequence, the method comprising
providing a first polypeptide-encoding nucleotide sequence from a first organism, wherein said polypeptide-encoding nucleotide sequence includes a plurality of codons encoding a polypeptide sequence;
identifying the frequency at which a first codon of said first polypeptide-encoding nucleotide sequence occurs in polypeptide-encoded genes of said first organism; and
replacing at least one nucleotide in said first codon, thereby producing a second nucleotide sequence including a first replacement codon, wherein said first replacement codon occurs at a different frequency in polypeptide-encoded genes of said first organism than said first codon,
thereby modifying a polypeptide-encoding nucleotide sequence.

21. The method of claim 20, wherein said first replacement codon occurs at a lower frequency in polypeptide-encoding genes of said first organism than said first codon.

22. The method of claim 20, wherein said first replacement codon occurs at a higher frequency in polypeptide-encoding genes of said first organism than said first codon.

23. The method of claim 20, wherein said first replacement codon encodes an amino acid identical to the amino acid encoded by said first codon.

24. The method of claim 20, wherein said method further comprises
identifying the frequency at which a second codon of said first polypeptide-encoding nucleotide sequence occurs in polypeptide-encoded genes of said first organism; and
replacing at least one nucleotide in said second codon, thereby producing a second nucleotide sequence including a second replacement codon, wherein said second replacement codon occurs at a different frequency in polypeptide-encoded genes of said first organism than said first codon.

25. The method of claim 24, wherein said second codon is adjacent to said first codon in said first polypeptide-encoding polynucleotide sequence.

26. The method of claim 20, wherein said second nucleotide sequence encodes an RNA molecule translated at a different rate than an RNA molecule encoded by said first polypeptide-encoding nucleotide sequence.

27. The method of claim 26, wherein said second nucleotide sequence encodes an RNA molecule that is translated more rapidly than said first polypeptide-encoding nucleotide sequence.

28. The method of claim 26, wherein said second nucleotide sequence encodes an RNA molecule that is translated more slowly than said first polypeptide-encoding nucleotide sequence.

29. The method of claim 20, further comprising
identifying the frequency at which said first codon occurs in polypeptide-encoded genes of a second organism; and
replacing at least one nucleotide in said first codon to produce a first replacement codon.

30. The method of claim 29, wherein said second codon occurs at a similar frequency in said second organism as the first codon occurs in the polypeptide-encoded genes of said first organism.

31. A method for modifying a polypeptide-encoding nucleotide sequence, the method comprising
providing a first polypeptide-encoding nucleotide sequence, wherein said polypeptide-encoding nucleotide sequence includes a plurality of codons encoding a polypeptide sequence;
identifying the guanine-cytosine content of a first codon in said polypeptide-encoding sequence;
replacing at least one nucleotide in said first codon, thereby producing a second nucleotide sequence including a first replacement codon, wherein said first replacement codon has a guanine-cytosine content different than said first codon, and wherein said first codon and said first replacement codon encode the same amino acids.

32. The method of claim 31, wherein said second polynucleotide sequence encodes an RNA molecule translated at a rate different than an RNA molecule encoded by said first polynucleotide sequence.

33. The method of claim 31, further comprising
identifying the guanine-cytosine content of a second codon in said polypeptide-encoding nucleotide sequence, and
replacing at least one nucleotide in said second codon, thereby producing a second nucleotide sequence including a second replacement codon, wherein said second replacement codon has a guanine-cytosine content different than said second codon, and wherein said second replacement codon and said second codon encode the same amino acids.

34. The method of claim 33, wherein said second codon is adjacent to said first codon.

35. A method for constructing a nucleic acid for increasing expression of a polypeptide-encoding nucleotide sequence, the method comprising
identifying codon frequencies of a polypeptide-encoding nucleotide sequence and codon frequencies in polypeptide-encoded genes of a first cell;
comparing said codon frequencies, thereby identifying at least one rare codon that is abundant in said transgene and occurs in low frequency in polypeptide-encoded genes of said cell; and
constructing an episomal vector comprising a tRNA gene with an anticodon for said rare codon, thereby constructing a nucleic acid for increasing expression of a polypeptide-encoding nucleotide sequence.

36. The method of claim 35, further comprising identifying codon frequencies of a second rare codon and constructing an episomal vector comprising a tRNA gene with an anticodon for said second rare codon.

37. The method of claim 33, wherein said host cell is a prokaryotic cell.

38. The method of claim 33, wherein said prokaryotic cell is an E. coli cell.

39. A cell that includes the episomal vector of claim 35.
